(19) United States
(12) Patent Application Publication
(10) Pub. No.: US 2025/0320332 A1
(43) Pub. Date: Oct. 16, 2025

(54) SUBSTANTIALLY SEQUENCE-UNIFORM ALIPHATIC COPOLYESTER AND METHOD OF MAKING THE SAME

(71) Applicant: Purdue Research Foundation, West Lafayette, IN (US)

(72) Inventors: You-Yeon Won, West Lafayette, IN (US); Jin Yoo, West Lafayette, IN (US); Samruddhi M. Patil, West Lafayette, IN (US)

(21) Appl. No.: 18/576,616

(22) PCT Filed: May 10, 2022
(86) PCT No.: PCT/US22/28607

§ 371 Date: Jan. 4, 2024

Related U.S. Application Data
(60) Provisional application No. 63/221,143, filed on Jul. 13, 2021.

(51) Int. Cl. C08G 63/08; A61K 9/107; A61K 9/51; C08G 63/82; C08G 63/85

(52) U.S. Cl. CPC C08G 63/08; A61K 9/1075; A61K 9/5153; A61K 9/5192; C08G 63/823; C08G 63/85

(57) ABSTRACT

Various aspects disclosed relate to a method of preparing a substantially sequence-uniform aliphatic copolyester. The method includes continuously contacting at least, a first monomer and a second monomer with an initiator and a catalyst to initiate ring-opening copolymerization of the first monomer and the second monomer. In the method the first monomer and the second monomer are contacted with the initiator and catalyst at a feed rate that is slower than a polymerization rate of the first monomer and the second monomer.

### CROSS-REFERENCE TO RELATED APPLICATIONS

This application claims the benefit of priority to U.S. Provisional Patent Application Ser. No. 62/211,143 entitled “STRATEGY FOR SYNTHESIS OF STATISTICALLY SEQUENCE-CONTROLLED UNIFORM PLGA, AND EFFECTS OF SEQUENCE DISTRIBUTION ON INTERACTION AND DRUG RELEASE PROPERTIES,” filed Jul. 13, 2021 the disclosure of which is incorporated herein in its entirety by reference.

### STATEMENT OF GOVERNMENT SUPPORT

This invention was made with government support under CBET-1803968 awarded by the National Science Foundation. The government has certain rights in the invention.

### BACKGROUND

Poly(lactic-co-glycolic acid) (PLGA) is frequently used in pharmaceutical applications. However, produced PLGA is not uniform in the distribution of the monomers across the polymer chain. A non-uniform distribution of the monomers can lead to poor performance, for example, poor performance of a nanoparticle including PLGA for drug release.

### SUMMARY OF THE INVENTION

Most notably, a substantially uniform PLGA exhibited the desired, more sustained drug release behavior, compared to gradient PLGA. Various aspects disclosed relate to a method of preparing a substantially sequence-uniform aliphatic copolyester. The method includes continuously contacting at least, a first monomer and a second monomer with an initiator and a catalyst to initiate ring-opening copolymerization of the first monomer and the second monomer. In the method the first monomer and the second monomer are contacted with the initiator and catalyst at a feed rate that is slower than a polymerization rate of the first monomer and the second monomer. This approach differs significantly from previous approaches where only the more reactive monomer is continuously added to compensate for its faster consumption with the less reactive monomer only added initially. The instant approach allows for simultaneous feeding of multiple monomers.

### BRIEF DESCRIPTION OF THE FIGURES

The drawings illustrate generally, by way of example, but not by way of limitation, various embodiments of the present invention.

FIG. 1 shows ¹³C NMR spectra (carbonyl resonances) of the PEG_{5.0k}-PL_{5.0k}G_{5.0k}A polymers produced under three different monomer feed rate conditions (named OLG1, OLG2, and OLG3).

FIG. 2A shows DSC curves for various components.

FIG. 2B shows TGA traces for various components.

FIG. 3A shows the thermo-responsive gelation behavior of a PEG-PLGA micelle.

FIG. 3B shows SEM images of a PTX-loaded PEG-PLGA nanoparticle.

FIG. 3C shows the kinetics of PTX release from a PEG-PLGA nanoparticle.

FIG. 4 is a schematic figure illustrating a summary of the effects of PLGA monomer sequence distribution on the conformational and interaction properties of aqueous PEG-PLGA self-assemblies.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Poly(lactic-co-glycolic acid) (PLGA) is one of the most widely used polymers in pharmaceutical applications. Studies have been conducted to elucidate the effects of such parameters as molecular weight, polydispersity and monomer composition on the controlled release properties of PLGA. However, studies dealing with the effect of monomer sequence distribution have been sparse because of the inability of controlling monomer sequence in PLGA using conventional batch ring-opening copolymerization processes.

The instant disclosure relates to a scalable semi-batch copolymerization protocol that results in the production of statistically sequence-controlled substantially “uniform PLGA” polymers through control of the rate of comonomer (lactide and glycolide) addition. Using this feed rate-controlled, semi-batch copolymerization method, a series of PEGylated PLGA (PEG-PLGA) samples having an identical molecular weight and monomer composition but different sequence distributions (uniform vs. gradient) were prepared. Key physicochemical properties of these materials were examined both in the neat state (PEG crystallization/melting, hygroscopicity) and in aqueous solution (sol-gel transition, drug release kinetics). All measured properties significantly varied among the samples, demonstrating that the implementation of comonomer sequence control only at the statistical level still significantly influences the properties of the copolymer products.

According to various aspects of the present disclosure, a method for preparing the substantially sequence-uniform aliphatic copolyester includes continuously contacting at least, a first monomer and a second monomer with an initiator and a catalyst to initiate ring-opening copolymerization of the first monomer and the second monomer. The first monomer and the second monomer are contacted with the initiator and catalyst at a feed rate that is slower than a polymerization rate of the first monomer and the second monomer. This method can be referred to as “feed rate-controlled polymerization”.

Mechanistically, feed rate-controlled polymerization functions such that the disparity in monomer reactivities becomes an unimportant factor in the slow co-monomer feed rate limit; when the feed rate is slower than the consumption (polymerization) rates of the monomers, the monomer sequence distribution of the copolymer becomes substantially uniform.

The first monomer and the second monomer can be co-dispensed from the same container. Alternatively, the first monomer and the second monomer can be located in separate containers and separately dispensed. In the example where the first monomer and the second monomer are located in separate containers, the feed rate of the first monomer and the second monomer can be substantially the same or can be substantially different feed rates.

The particular feed rate of the first monomer and the second monomer can depend on various factors such as the volume of the container to which the first monomer and the second monomer are dispensed. As an example, a feed rate of the first monomer and the second monomer (collectively the “comonomers”) can be in a range of from about 5.0×10⁻⁶ moles of comonomers/min per mole of catalyst to about 5.0×10¹ moles of comonomers/min per mole of catalyst, from about 5.0×10⁻⁵ moles of comonomers/min per mole of catalyst to about 5.0×10² moles of comonomers/min per mole of catalyst, from about 5.0×10⁻⁴ moles of comonomers/min per mole of catalyst to about 5.0×10³ moles of comonomers/min per mole of catalyst or from about 5.0×10⁻¹ moles of comonomers/min per mole of catalyst to about 5.0 moles of comonomers/min per mole of catalyst.

According to various aspects, the aliphatic copolyester formed is poly(lactic-co-glycolic acid). The various monomers used can include glycolide (GL), lactide (LA). In some examples the first monomer is lactide and the second monomer is glycolide. In some further examples, the method can include co-dispensing a third monomer along with the first monomer and the second monomer. In such an example, the third monomer can be dispensed at the same feed rate as the first monomer, the second monomer, or both.

Any of the first monomer, second monomer, third monomer, or mixture thereof can be dispensed in a solvent prior to contact with the catalyst and initiator. The solvent can be an organic solvent such as dichloromethane. In some examples, the first monomer, second monomer, third monomer, or mixture thereof are not dispersed in a solvent (e.g., substantially free of a solvent or using one or two monomers as a solvent).

Similarly, the initiator, catalyst, or both can be dispersed in a solvent or not. The initiator and catalyst can be disposed together in the same container or vessel and in direct fluid contact with the first monomer, second monomer, third monomer, or a mixture thereof (e.g., in a syringe pump, where a mixture comprising the initiator and/or catalyst is provided through the needle to a reactor comprising the first monomer, second monomer or mixtures thereof).

The initiator (a source of any chemical species that reacts with a monomer to form an intermediate compound capable of linking successively with a large number of other monomers into a polymeric compound) is an alcohol. An example of a suitable alcohol is a glycol such as polyethylene glycol (R—(O—CH₂—CH₂)ₙ—OH where typically R═H or CH₃). A weight-average molecular weight of the polyethylene glycol can be in a range of from about 100 g/mol to about 1×10⁶ g/mol.

The catalyst reduces the activation energy required to effect polymerization. Examples of catalysts that can be used include an organic amidine compound, an organic guanidine compound, an aminopyridine compound, a thiourea compound, a heterocyclic carbene compound, a tin-containing compound, or a mixture thereof. As an example, the organic amidine compound comprises 1,8-diazabicyclo[5.4.0]undec-7-ene (DBU). As an example, the organic guanidine compound comprises 1,5,7-triazabicyclo[4.4.0]dec-5-ene (TBD). As an example, the tin-containing compound comprises stannous octoate. Other examples of suitable catalysts include organic catalysts such as other amidines (e.g., 1,5-diazabicyclo[4.3.0]non-5-ene (DBN)), other guanidines (e.g., N-methyl-1,5,7-tri-azabicyclododecene (MTBD)), aminopyridines (e.g., 4-(dimethylamino)pyridine (DMAP)), thioureas (thioimidates), and N-heterocyclic carbenes.

As mentioned herein, the aliphatic copolyester formed can be a poly(lactic-co-glycolic acid). According to various examples, the produced aliphatic polyester can be subjected to a pre- or post-polymerization PEGylation process. The produced polymer can be characterized by its molecular weight distribution polydispersity index, which is in the range of from 1.0 and 3.0 or 1.0 to 2.5. In analyzing the structure of the produced polymer, the monomers are substantially uniformly distributed about the polymer molecule. That is, the produced polymer (uniform copolymer) is apparently similar to, but conceptually different from, a random copolymer where monomer residues are located randomly in the polymer molecule, because uniform copolymer is produced from comonomers, which have very disparate reactivities, using a semibatch comonomer addition method. As an example, the produced polymer can be a statistically alternating copolymer.

As understood herein, a random copolymer is a different concept than “uniform copolymer”. A random copolymer is a copolymer in which the composition of the copolymer is constant throughout the polymerization (for example, because it is produced at an azeotrope and thus contains no composition drift) and equal to the monomer composition in the reactor (F1=f1); a random copolymer is an idealized material that can only be produced when the reactivity ratios of the two monomers are both equal to unity (r1=r2=1). Therefore, for instance, random PLGA is not conceptually identical to uniform PLGA because uniform PLGA is produced from the two monomers, LA and GL, which have very disparate reactivities, using a semibatch, comonomer addition method.

The aliphatic polyester formed according to the instantly described methods can have the advantage of being used to form a nano- or microparticle. The nano- or microparticle, for example, is useful for drug delivery in that is shows favorable drug loading and release characteristics. Examples of suitable drugs that can be loaded in the nanoparticle can include paclitaxel, docetaxel, leuprolide acetate, goserelin acetate, octreotide acetate, somatotropin, triptorelin pamoate, lanreotide, minocycline HCl, risperidone, naltrexone, dexamethasone, mometasone furoate, exenatide, pasireotide, triamcinolone acetoamide, buprenorphine. The drug can be in a range of from about 0.5 wt % to about 50 wt % of the nano- or microparticle.

The nanoparticle can have a substantially spherical shape. An average diameter of the nano- or microparticle can be in a range of from about 20 nm to about 2000 nm or about 200 nm to about 2×10⁵ nm. The pharmaceutical component can be distributed about or doped in the nanoparticle.

### EXAMPLES

Various embodiments of the present invention can be better understood by reference to the following Examples which are offered by way of illustration. The present invention is not limited to the Examples given herein.

### EXPERIMENTAL PROCEDURES

Chemicals. Rac-lactide (LA), mPEG-OH (Mₙ=5,000 g/mol), benzoic acid, dichloromethane (DCM, anhydrous), 1,8-diazabicyclo[5.4.0]undec-7-ene (DBU), and deuterated dimethylsulfoxide (DMSO-d₆) were purchased from Sigma-Aldrich. Glycolide (GL) and hexafluoroisopropanol were purchased from TCI America. CDCl₃ was purchased from Cambridge Isotope Laboratories. Laponite® was purchased from BYK USA, Inc. A commercial PEG_{5.0k}-PL_{5.0k}G_{5.0k}A material was purchased from PolySciTech Division of Akina, Inc. (Catalog No. AK010, Lot No. 180615RAI-A). Paclitaxel (PTX) was purchased from Samyang Biopharmaceuticals.

Synthesis of PEG-PLGA copolymers. All reactions were carried out in oven-dried glassware under nitrogen atmosphere using the standard Schlenk line technique at room temperature unless specified otherwise. A comonomer solution was prepared by dissolving designated amounts of LA and GL (comonomers) in 10.0 mL of anhydrous DCM and charged into a 10-mL plastic Norm-Ject syringe. 335 mg of mPEG-OH (initiator) was dissolved in 5.0 mL of anhydrous DCM within an oven-dried round-bottom flask containing a magnetic stirring bar and capped with a rubber septum. A designated volume of DBU (catalyst) was dissolved in 1.0 mL of anhydrous DCM within a screw-cap vial. After adding the DBU solution to the mPEG-OH solution inside the reactor, a syringe pump setup was arranged for injection of the comonomer solution into the reactor. The comonomer solution was injected at a constant, specified rate. At the end of the comonomer injection period (which was equal to the comonomer solution volume(=10.0 mL) divided by the comonomer solution injection rate), excess benzoic acid (200 mg) was added to the reaction mixture to terminate the polymerization process. The final reaction mixture was cast into cold isopropanol and centrifuged to collect the polymer product as a precipitate. The polymer product was dried under vacuum overnight.

Characterization of PEG-PLGA copolymers. ¹H and ¹³C NMR measurements were performed using a Bruker AVANCE III 400 MHz NMR spectrometer. Chemical shifts were recorded in ppm with reference to solvent signals. From ¹H NMR spectra (obtained using CDCl₃ as the solvent), the number-average molecular weights (Mₙ's) of the PLGA blocks were determined; the combined area under the lactate's methine and glycolate's methylene peaks of PLGA (5.2 and 4.8 ppm, respectively) was compared to the area under the methylene peak of PEG (3.6 ppm) in order to determine the Mₙ of the PLGA block on the basis of the known value of Mₙ for the PEG precursor (5,000 g/mol, information provided by the vendor). From ¹³C NMR spectra (i.e., the carbonyl signals of PLGA obtained using either DMSO-d₆ or hexafluoroisopropanol (used in a coaxial tube insert) as the solvent), the cumulative number-average lactate and glycolate sequence lengths were determined; the coaxial-tube measurements performed in hexafluoroisopropanol used a coaxial NMR tube outer insert and DMSO-d₆ for an internal solvent lock and chemical shift referencing. In ¹³C NMR measurements, the number of transients was 1,000, and a relaxation delay of 6 s was used.

Differential scanning calorimetry (DSC) measurements were performed using a Perkin Elmer DSC 4000 instrument. Approximately 6 mg of polymer was loaded into a hermetically sealed aluminum pan for a DSC experiment. All measurements were performed under gentle nitrogen purge.

Thermogravimetric analysis (TGA) measurements were performed using a TA Instruments Q600 SDT instrument. Immediately prior to TGA analysis, a polymer specimen placed in a petri dish was kept under 85% relative humidity (controlled by supersaturated KCl) at ambient temperature inside a closed chamber for 2 days. All measurements were performed under helium environment at a heating rate of 10° C./min.

Gel permeation chromatography (GPC) experiments were performed with a Waters Breeze HPLC system equipped with an isocratic pump, Styragel HR 4 (10⁴ Å pore size) and Ultrastyragel (500 Å pore size) columns (7.8×300 mm per column) and a differential refractometer using tetrahydrofuran (THF) as the mobile phase at a flow rate of 1.0 mL/min at 30° C. 20 μL of a 3 mg/mL polymer solution in THF was injected into the GPC system, and the refractive index signal was recorded.

Derivation of equations used for calculation of monomer sequence lengths from ¹³C NMR data. Within the terminal model, the normalized instantaneous probability of finding k units of monomer i is given by:

$${S}_{i , k}^{inst} = {P}_{ii}^{k - 1} ( 1 - {P}_{ii} ) \qquad ( 1 )$$

where Pᵢⱼ is the probability of adding monomer j to a chain end containing monomer i (i, j=1 (LA) or 2 (GL)). The instantaneous relative molar concentrations of lactate-lactate-lactate, lactate-lactate-glycolate (or glycolate-lactate-lactate), glycolate-glycolate-lactate (or lactate-glycolate-glycolate), and glycolate-glycolate-glycolate triads

$${I}_{111}^{inst} , {I}_{112}^{inst} ( = {I}_{211}^{inst} ) , {I}_{221}^{inst} ( = {I}_{122}^{inst} ) , and {I}_{222}^{inst} ,$$

respectively) are related to these instantaneous sequence probabilities by

$${I}_{111}^{inst} ∝ ( 0 \cdot {S}_{1 , 1}^{inst} + 2 \cdot {S}_{1 , 2}^{inst} + 4 \cdot {S}_{1 , 3}^{inst} + … ) \qquad ( 2 )$$

$${I}_{112}^{inst} ( = {I}_{211}^{inst} ) ∝ ( 2 \cdot {S}_{1 , 1}^{inst} + 2 \cdot {S}_{1 , 2}^{inst} + 2 \cdot {S}_{1 , 3}^{inst} + … ) \qquad ( 3 )$$

$${I}_{221}^{inst} ( = {I}_{122}^{inst} ) ∝ ( 2 \cdot {S}_{2 , 1}^{inst} + 2 \cdot {S}_{2 , 2}^{inst} + 2 \cdot {S}_{2 , 3}^{inst} + … ) \qquad ( 4 )$$

$${I}_{222}^{inst} ∝ ( 0 \cdot {S}_{2 , 1}^{inst} + 2 \cdot {S}_{2 , 2}^{inst} + 4 \cdot {S}_{2 , 3}^{inst} + … ) . \qquad ( 5 )$$

Substitution of Eq. (1) into Eqs. (2)-(5) gives

$${I}_{111}^{inst} = ( const ) {\sum }_{k = 1}^{∞} ( 2 k - 2 ) {P}_{11}^{k - 1} ( 1 - {P}_{11} ) = 2 ( const ) \frac{{P}_{11}}{1 - {P}_{11}} \qquad ( 6 )$$

$${I}_{112}^{inst} ( = {I}_{211}^{inst} ) = 2 ( const ) {\sum }_{k = 1}^{∞} {P}_{11}^{k - 1} ( 1 - {P}_{11} ) = 2 ( const ) \qquad ( 7 )$$

$${I}_{221}^{inst} ( = {I}_{122}^{inst} ) = 2 ( const ) {\sum }_{k = 1}^{∞} {P}_{22}^{k - 1} ( 1 - {P}_{22} ) = 2 ( const ) \qquad ( 8 )$$

$${I}_{222}^{inst} = ( const ) {\sum }_{k = 1}^{∞} ( 2 k - 1 ) {P}_{22}^{k - 1} ( 1 - {P}_{22} ) = 2 ( const ) \frac{{P}_{22}}{1 - {P}_{22}} \qquad ( 9 )$$

From Eqs. (6) and (7), one obtains

$${P}_{11} = \frac{{I}_{111}^{inst}}{{I}_{111}^{inst} + {I}_{112}^{inst}} . \qquad ( 10 )$$

From Eqs. (8) and (9), one obtains

$${P}_{22} = \frac{{I}_{222}^{inst}}{{I}_{222}^{inst} + {I}_{221}^{inst}} . \qquad ( 11 )$$

Therefore, the instantaneous number- and weight-average repeat unit sequence lengths can be calculated as

$${( {n}_{1} )}_{n}^{inst} = 2 {( {N}_{1} )}_{n}^{i n s t} = \frac{2}{1 - {P}_{11}} = 2 \frac{{I}_{111}^{inst}}{{I}_{112}^{inst}} + 2 \qquad ( 12 )$$

$${( {n}_{2} )}_{n}^{inst} = 2 {( {N}_{2} )}_{n}^{i n s t} = \frac{2}{1 - {P}_{22}} = 2 \frac{{I}_{222}^{inst}}{{I}_{221}^{inst}} + 2 \qquad ( 13 )$$

$${( {n}_{1} )}_{w}^{i n s t} = 2 {( {N}_{1} )}_{w}^{i n s t} = \frac{1 + {P}_{11}}{1 - {P}_{11}} = 4 \frac{{I}_{111}^{lnst}}{{I}_{112}^{inst}} \qquad ( 14 )$$

$${( {n}_{2} )}_{w}^{i n s t} = 2 {( {N}_{2} )}_{w}^{i n s t} = \frac{1 + {P}_{22}}{1 - {P}_{22}} = 4 \frac{{I}_{222}^{lnst}}{{I}_{221}^{inst}} \qquad ( 15 )$$

Note the lactate (or glycolate) repeat unit sequence length (“nᵢ”) is twice the LA (or GL) monomer sequence length (“Nᵢ”), because when polymerized, each LA (or GL) monomer turns into two lactate (or glycolate) repeat units. Typically, ¹³C NMR measurements are performed on final products of polymerization, which give data for the cumulative (instead of instantaneous) relative dyad concentrations

$$( {I}_{111}^{c u m u} , {I}_{112}^{c u m u} ( = {I}_{211}^{c u m u} ) , {I}_{221}^{c u m u} ( = {I}_{122}^{c u m u} ) , and {I}_{222}^{c u m u} ) .$$

The cumulative number- and weight-average repeat unit sequence lengths can be calculated from the NMR results using the following pre-averaging approximations (analogous to Eqs. (12)-(15)):

$${( {n}_{1} )}_{n}^{c u m u} = 2 {( {N}_{1} )}_{n}^{c u m u} ≅ 2 \frac{{I}_{111}^{c u m u}}{{I}_{112}^{c u m u}} + 2 \qquad ( 16 )$$

$${( {n}_{2} )}_{n}^{c u m u} = 2 {( {N}_{2} )}_{n}^{c u m u} ≅ 2 \frac{{I}_{222}^{c u m u}}{{I}_{221}^{c u m u}} + 2 \qquad ( 17 )$$

$${( {n}_{1} )}_{w}^{c u m u} = 2 {( {N}_{1} )}_{w}^{c u m u} ≅ 4 \frac{{I}_{111}^{c u m u}}{{I}_{112}^{c u m u}} \qquad ( 18 )$$

$${( {n}_{2} )}_{w}^{c u m u} = 2 {( {N}_{2} )}_{w}^{c u m u} ≅ 4 \frac{{I}_{222}^{c u m u}}{{I}_{221}^{c u m u}} \qquad ( 19 )$$

Eqs. (16) and (17) were used to calculate the cumulative number- and weight-average lactate and glycolate sequence length values

$$\overline{{L}_{L}} ( = {( {n}_{1} )}_{n}^{c u m u} ) and \overline{{L}_{G}} ( = {( {n}_{2} )}_{n}^{c u m u} ) ,$$

respectively) shown in Table 2 below. In the PLGA literature, the following simplified (dyadic) notations are commonly used for ¹³C NMR peak assignments:

$${I}_{111}^{c u m u} \to “ {I}_{11} ” , {I}_{112}^{c u m u} ( = {I}_{211}^{c u m u} ) \to “ {I}_{12} ” , {I}_{221}^{c u m u} ( = {I}_{122}^{c u m u} ) \to “ {I}_{21} ” , and {I}_{222}^{cumu} \to “ {I}_{22} ” .$$

Preparation of PTX-loaded PEG-PLGA nanoparticles (PEG-PLGA/PTX NPs) via an emulsion-evaporation process. 40 mg of PEG-PLGA and 4 mg of PTX were dissolved in 2 mL of DCM (organic phase). 400 mg of PVA (emulsifier, Mₙ=124,000 g/mol, Acros) was dissolved in 20 mL of Milli-Q water initially at 80° C., and the solution was cooled down to room temperature (aqueous phase). The organic phase was added to the aqueous phase, and the mixture was emulsified using a high-speed disperser (T25 Digital Ultra-Turrax®, IKA, Germany) at 22,000 rpm for 8 min to form an O/W emulsion. The organic solvent was evaporated under atmospheric pressure at room temperature overnight while the solution was kept under magnetic stirring. The resultant PTX-loaded PEG-PLGA nanoparticles were washed with Milli-Q water and collected by centrifugation at 8,000 rpm for 7 min; this washing process was repeated 4 times to remove PVA.

Characterization of PTX-loaded PEG-PLGA nanoparticles. The morphologies of PTX-loaded PEG-PLGA nanoparticles were investigated by scanning electron microscopy (SEM) (Nova NanoSEM 450, FEI) and transmission electron microscopy (TEM) (Tecnai T20, FEI). For SEM, an approximately 20 μL drop of 0.5 mg/mL PEG-PLGA/PTX NPs in Milli-Q water was placed on a Si wafer, dried in air, and coated with Pt under vacuum. For TEM, an approximately 20 μL drop of 0.5 mg/mL PEG-PLGA/PTX NPs in Milli-Q water was placed on a plasma-cleaned, carbon-coated Formvar TEM grid, dried in air, and stained with 2% uranyl acetate as a negative staining agent.

Dynamic light scattering (DLS) was used to determine the mean hydrodynamic diameter and polydispersity index (PDI) of PEG-PLGA micelles and PEG-PLGA/PTX NPs dispersed in aqueous media. Measurements (N=5) were performed using a NanoBrook Omni instrument (Brookhaven Instruments), which operates at an incident light wavelength of 640 nm and a scattering angle of 90°.

The PTX encapsulation efficiency (EE for short, defined as the mass of PTX encapsulated divided by the mass of PTX initially added) and loading content (LC, defined as the mass of PTX encapsulated divided by the total mass of PTX and polymer in the nanoparticle) were determined using an isocratic reverse phase HPLC method. Experiments were performed as follows. 20 mg of purified and dried PEG-PLGA/PTX NPs was dissolved in 10 mL of acetonitrile (ACN); the mixture was vortexed until it became transparent. This solution was analyzed by HPLC (HP 1100, Agilent Technologies) to determine the concentration of PTX. A C18 column (4×125 mm) was used as the stationary phase. A 45:55 by volume mixture of water and acetonitrile (containing 0.1% (v/v) formic acid) was used as the mobile phase at a flow rate of 1.0 mL/min. The PTX absorbance was measured at 227 nm wavelength.

The kinetics of PTX release from PEGA-PEG/CWO/PTX NPs were measured as follows. 10 mg of purified and dried PEG-PLGA/CWO/PTX NPs were re-dispersed in 1.0 mL PBS buffer containing 0.1% (v/v) Tween 80 (release medium), and the solution was kept at 37° C. under constant magnetic stirring (N=3 for each sample). At predetermined times, the supernatant was sampled using the following procedure: (i) the solution was centrifuged at 8,000 rpm for 7 min at room temperature to separate the NP pellet from the supernatant; (ii) 0.8 mL of the supernatant was collected; (iii) the same volume (0.8 mL) of fresh release medium was added to the remaining supernatant; (iv) the NP pellet was re-dispersed by shaking; (v) the solution was further incubated at 37° C. under magnetic stirring. The sampled supernatant was analyzed by HPLC using the procedure described above to determine its PTX content.

Preparation and characterization of PEG-PLGA micelle and laponite mixtures in water. 40 mg of PEG-PLGA was dissolved by direct hydration in 2 mL of Milli-Q water. 40 mg of laponite was dispersed in 2 mL of Milli-Q water. These two solutions were mixed at a ratio of PEG-PLGA:laponite=1:3 by volume (i.e., 0.5 mL of the PEG-PLGA solution+1.5 mL of the laponite solution); the final solution contained 0.5% (w/v) PEG-PLGA and 1.5% (w/v) laponite. The sol-gel transition temperature of this PEG-PLGA/laponite solution was determined via vial inversion assay. 2.0 mL of the solution was placed in a 4-mL vial, and the vial was immersed in a temperature-regulated oil bath. The gelation test was performed while the temperature of the bath was increased by a 1° C. increment. The solution was considered to be gelled if the solution did not flow for 30 s upon inversion of the vial.

To demonstrate the FRCP concept, a series of PEG_{5.0k}-PL_{2.5k}G_{2.5k}A materials were synthesized under different combinations of comonomer feed rate and polymerization rate conditions; here, the polymerization rate was controlled by catalyst (DBU) concentration. Briefly, 10 mL of a comonomer solution containing 116 mM LA and 144 mM GL in dichloromethane (DCM) was injected at various rates (0.05-0.3 mL/min) into the reactor that initially contained 5 mL of an initiator/catalyst solution containing 11.2 mM mPEG-OH (initiator) and 11-33 mM DBU (catalyst) in DCM. The polymerization was run for the period of time needed to complete the comonomer injection (e.g., for 100 minutes at a monomer feed rate of 0.10 mL/min) and then terminated by adding excess benzoic acid. As summarized in Table 1 (Runs 1-5), at fast monomer feed rates and/or low DBU concentrations (slow polymerization rates), the reaction mixture turned opaque due to the generation of (PEG-)PLGA chains containing long sequences of GL monomers and their precipitation from DCM; at slower monomer feed rates/higher DBU concentrations, the reaction mixture remained transparent throughout the polymerization process, suggesting more substantially uniform sequence characteristics for the PLGA products. The resultant PEG-PLGA was cast into 200 mL of ice-cold isopropanol, which is a nonsolvent for PEG and PLGA (to remove soluble benzoic acid and LA residues), collected as a precipitate via centrifugation, dried, re-dissolved in CDCl₃, and characterized by ¹H NMR to determine its molecular weight. As shown in Table 1, the unprecipitated reaction products (Runs 2 and 3 in Table 1) showed PLGA block molecular weights (˜4.0 kDa) that are about 20% less than the target value (5.0 kDa); as the monomer feed rate was increased (Runs 4 and 5) or the DBU concentration was decreased (Run 1), the PLGA block molecular weight further decreased because of the precipitation of the growing chains which limited the polymerization conversion.

TABLE 1
Summary of PEG-PLGA polymers synthesized using the FRCP method under different comonomer feed rate
and DBU concentration conditions. The reaction conditions were as follows: for all runs, volume of
comonomer feed solution injected into reactor = 10 mL, initial volume of initiator/catalyst solution in reactor =
6.0 mL, [mPEG-OH]ₒ = 11.2 mM (reactor), solvent = DCM (for both comonomer and initiator/catalyst
solutions), T = 25° C.; for PEG_{5.0k}-PL_{2.5k}G_{2.5k}A, [LA]ₒ = 116 mM (feed), [GL]ₒ = 144 mM (feed); for
PEG_{5.0k}-PL_{5.0k}G_{5.0k}A, [LA]ₒ = 232 mM (feed), [GL]ₒ = 289 mM (feed); for PEG_{5.0k}-PL_{7.5k}G_{2.5k}A,
[LA]ₒ = 349 mM (feed), [GL]ₒ = 144 mM (feed).

| Run No. | Polymer Target | Feed Rate (mL/min) | [DBU]ₒ (IM) | Product in DCM. | Polymer Product ^{b} |
|---|---|---|---|---|---|
| 1 | PEG_{5.0k}-PL_{2.5k}G_{2.5k}A | 0.10 | 11.1 | Opaque | PEG_{5.0k}-PL_{0.29k}G_{1.2k}A |
| 2 | PEG_{5.0k}-PL_{2.5k}G_{2.5k}A | 0.05 | 33.5 | Transparent | PEG_{5.0k}-PL_{1.5k}G_{2.1k}A |
| 3^{a} | PEG_{5.0k}-PL_{2.5k}G_{2.5k}A | 0.10 | 33.5 | Transparent | PEG_{5.0k}-PL_{2.0k}G_{2.0k}A |
| 4 | PEG_{5.0k}-PL_{2.5k}G_{2.5k}A | 0.20 | 33.5 | Translucent | PEG_{5.0k}-PL_{1.9k}G_{2.0k}A |
| 5 | PEG_{5.0k}-PL_{2.5k}G_{2.5k}A | 0.30 | 33.5 | Opaque | PEG_{5.0k}-PL_{1.7k}G_{2.0k}A |
| 6^{a} | PEG_{5.0k}-PL_{2.5k}G_{2.5k}A | 0.05 | 33.5 | Transparent | PEG_{5.0k}-PL_{4.3k}G_{2.5k}A |
| 7 | PEG_{5.0k}-PL_{2.5k}G_{2.5k}A | 0.10 | 33.5 | Opaque | PEG_{5.0k}-PL_{4.1}kG_{4.6k}A |
| 8 | PEG_{5.0k}-PL_{2.5k}G_{2.5k}A | 0.03 | 33.5 | Transparent | PEG_{5.0k}-PL_{3.3k}G_{4.8k}A |
| 9 | PEG_{5.0k}-PL_{2.5k}G_{2.5k}A | 0.05 | 33.5 | Transparent | PEG_{5.0k}-PL_{5.6k}G_{1.7k}A |
| 10^{a} | PEG_{5.0k}-PL_{2.5k}G_{2.5k}A | 0.05 | 55.8 | Transparent | PEG_{5.0k}-PL_{6.4k}G_{2.0k}A |
| 11 | PEG_{5.0k}-PL_{2.5k}G_{2.5k}A | 0.05 | 89.3 | Transparent | PEG_{5.0k}-PL_{5.5k}G_{2.0k}A |

^{a}Italicized entries represent the best among tested conditions identified for producing the respective target PEG-PLGA products.
^{b} Based on ¹H NMR.

Similar experiments have also been performed targeting PEG_{5.0k}-PL_{5.0k}G_{5.0k}A and PEG_{5.0k}-PL_{7.5k}G_{2.5k}A microstructures (Runs 6 and 7, and Runs 8-11, respectively, in Table 1). The comonomer feed volume and the initial initiator concentration were kept the same as in the previous series (10 mL and 33.5 mM, respectively); therefore, the total LA+GL concentration in the feed solution had to be doubled. Note for the PEG_{5.0k}-PL_{7.5k}G_{2.5k}A experiments, larger amounts of DBU had to be added in order to accelerate the rate of polymerization (i.e., to obtain a high monomer conversion) because in these experiments the less reactive LA was the majority monomer component; see Run 10. Overall, all qualitative trends were the same as those observed in the previous PEG_{5.0k}-PL_{2.5k}G_{2.5k}A case.

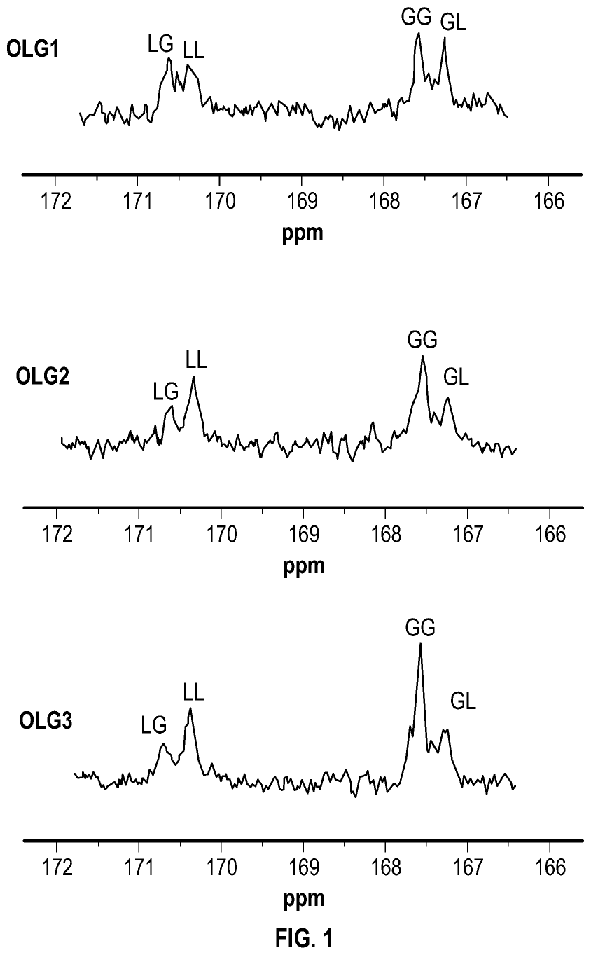

The PEG_{5.0k}-PL_{5.0k}G_{5.0k}A samples (Runs 6 and 7 in Table 1) were chosen for detailed microstructural investigation. As summarized in Table 2, the average sequence length properties of the copolymers in this family were measured to demonstrate the effect of comonomer feed rate on the sequence properties of the copolymer (the slower the comonomer feed rate is, the more uniform the copolymer sequence distribution becomes). Of note, in addition to the products of Runs 6 and 7 in Table 1 (named, respectively, as OLG2 and OLG3 in Table 2), one additional sample (OLG1) was prepared at a slower feed rate (0.03 mL/min) and included in this study; as indicated in Table 2, for the synthesis of the OLG1, DBU had to be injected multiple (three) times (20 μL of DBU injected at every 2 hours) in order to obtain a high monomer conversion because of the long reaction time(˜6 hours at the 0.03 mL/min comonomer addition rate), and the deactivation of DBU that occurs over time due to a trace amount of acid impurities. The monomer sequence properties of the OLG1, OLG2 and OLG3 samples were characterized by ¹³C NMR. The signals from the carbonyl carbons were analyzed to determine the cumulative relative lactyl-lactyl, lactyl-glycolyl, glycolyl-lactyl, and glycolyl-glycolyl diad concentrations (I_{LL}, I_{LG}, I_{GL}, and I_{GG}, respectively); the carbonyl signals were used because they are more sensitive to the sequence environment than the methyl, methylene and methine signals. The I_{LL} and I_{LG} data used in this analysis were obtained using hexafluoroisopropanol as the solvent (FIG. 1), and the I_{GL} and I_{GG} data used were obtained using DMSO-d₆ as the solvent. From the I_{LL}, I_{LG}, I_{GL} and I_{GG} values, the cumulative number-average lactate/glycolate repeat unit sequence lengths (L̅_̅{̅L̅}̅ and L̅_̅{̅G̅}̅, respectively) were calculated using the following equations

$$\vec{{L}_{L}} ≅ 2 \frac{{I}_{LL}}{{I}_{LG}} + 2 \qquad ( 1 )$$

$$\vec{{L}_{G}} ≅ 2 \frac{{I}_{GG}}{{I}_{GL}} + 2. \qquad ( 2 )$$

As shown in Table 2, the results confirm that a slower comonomer feed rate gives shorter average LA/GL sequence lengths and thus a more substantially uniform monomer sequence distribution.

TABLE 2
Cumulative number-average lactate and glycolate sequence lengths (L̅_̅{̅L̅}̅ and L̅_̅{̅G̅}̅, respectively)
for PEG_{5.0k}-PL_{5.0k}G_{5.0k}A polymers synthesized under different comonomer feed rate conditions.

| | Feed Rate | [DBU] [symbol] | Polymer | | | | |
|---|---|---|---|---|---|---|---|
| Polymer ID | (mL/min) | (mM) | Product ^{a} | I_{LL}/I_{LG} ^{b} | L̅_̅{̅L̅}̅ ^{c} | I_{GG}/I_{GL} ^{d} | L̅_̅{̅G̅}̅ ^{e} |
| OLG1 | 0.03 | 22.3 × 3^{e} | PEG_{5.0k}-PL_{5.2k}G_{5.0k}A | 1.48 | 4.96 | 1.89 | 5.78 |
| OLG2 | 0.05 | 33.5 | PEG_{5.0k}-PL_{4.3k}G_{4.6k}A | 1.78 | 5.56 | 2.06 | 6.12 |
| OLG3 | 0.10 | 33.5 | PEG_{5.0k}-PL_{4.1k}G_{4.8k}A | 1.74 | 5.48 | 2.58 | 7.16 |

^{a} Number-average molecular weight values were determined by ¹H NMR.
^{b} Determined by ¹³C NMR using hexafluoroisopropanol as the solvent (FIG. 1).
^{c} Calculated using Eqs (1) and (2).
^{d} Determined by ¹³C NMR using DMSO-d₆ as the solvent
^{e} “×3” denotes that 3 doses of DBU (20 μL of DBU per injection) were added to the reactor (at the beginning and every 2 hours thereafter). [symbol]
indicates data missing or illegible when filed

The molecular characteristics of the polymers produced by the FRCP method (OLG1, OLG2, and OLG3) were also compared with those of a commercial PEG_{5.0k}-PL_{5.0k}G_{5.0k}A product synthesized by a batch reaction with a tin (stannous octoate) catalyst (Catalog No. AK010, Lot No. 180615RAI-A, PolySciTech Division of Akina, Inc.). The number-average lactate/glycolate repeat unit sequence lengths of AK010 were determined by ¹³C NMR to be L̅_̅{̅L̅}̅≅4.76 and L̅_̅{̅G̅}̅≅5.98, respectively; in terms of sequence uniformity, AK010 is not better than OLG1 (Table 2), although in the AK010 case both the less disparate reactivities of LA and GL under the influence of tin and the transesterification that occurs after polymerization must have contributed to enhancing the uniformity of the monomer sequence distribution. These results demonstrate advantages of using the DBU-catalyzed FRCP method for producing substantially uniform PLGA products.

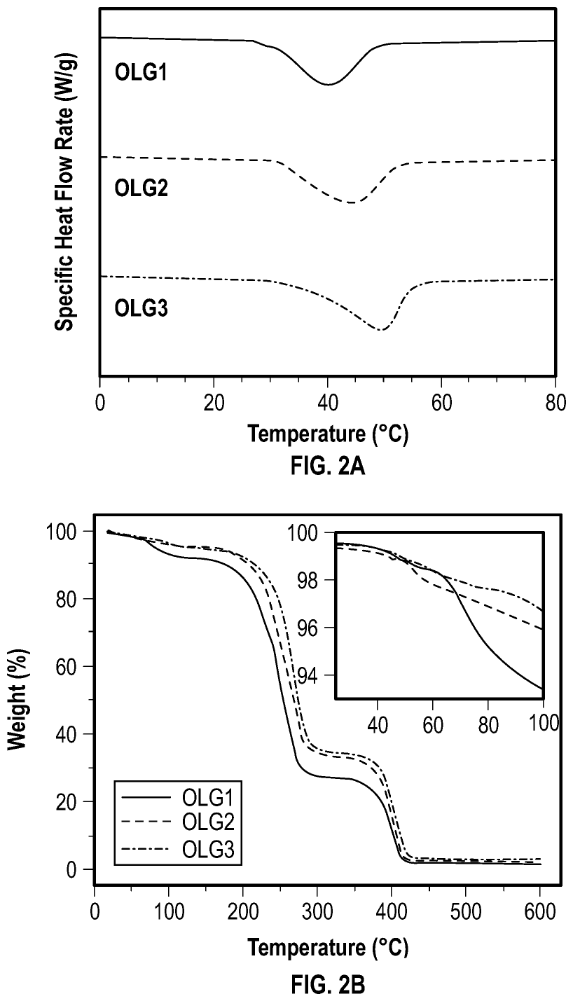

In the remainder of this Example, results are shown for investigating the effects of monomer sequence distribution on the structural and interaction properties of PEG-PLGA polymers. FIG. 2a shows differential scanning calorimetry (DSC) profiles of the 3 different FRCP products, OLG1, OLG2, and OLG3. Interestingly, the melting temperatures of the PEG_{5.0k}-PL_{5.0k}G_{5.0k}A copolymers (Tₘ(defined as the temperature at which the highest heat flow is observed)≅40-52° C.) were found to be significantly lower than that of mPEG_{5.0k}-OH (Tₘ≅67° C.). Among the three copolymer samples, the Tₘ decrease was greatest for OLG1 (Tₘ≅40° C.), followed by OLG2 (Tₘ≅44° C.) and then by OLG3 (Tₘ≅50° C.). It is believed that this trend reflects the fact that as one goes from OLG3 to OLG2 to OLG1, the monomer sequence gradient decreases, particularly near the junction point between the PEG and PLGA blocks. This can further be explained as follows. According to the Gibbs-Thomson equation, the melting temperature depression

$$( Δ {T}_{m} ≡ {T}_{m}^{∞} - {T}_{m}$$

where

$${T}_{m}^{∞}$$

is the equilibrium melting temperature) is linearly proportional to the interfacial tension (γ) between the crystalline and amorphous phases within the semi-crystalline polymer (PEG) region. This PEG crystal-melt interfacial tension (γ) is influenced by the miscibility between the PEG and PLGA blocks. Based on the solubility parameter values of PEG, PLA and PGA (δ_{PEG}≅19.2 (J/cc)^{1/2}, δ_{PLA}≅21.4 (J/cc)^{1/2}, δ_{PGA}≅23.8 (J/cc)^{1/2}) and the monomer volume of PEG (ν_{PEG}≅39.0 cc/mol), the Flory-Huggins interaction parameters are estimated to be χ_{PEG/PLA}≅0.18 and χ_{PEG/PGA}≅0.53, respectively, for the PEG/PLA and PEG/PGA combinations at 25° C., which implies that PEG interacts more favorably with PLA than it does with PGA. OLG1 contains the least amount of GL sequences near the block junction, and therefore the greatest amount of PLGA segments would be able to intrude into the fold region of the semi-crystalline PEG domain of the neat OLG1 material; OLG3 would be the opposite end of this comparison. Therefore, OLG1, followed by OLG2 and OLG3, should exhibit the highest γ and thus the lowest Tₘ (the greatest ΔTₘ) as observed experimentally. This trend observed in terms of ΔTₘ (FIG. 2a) also agrees with the trend in the degree of crystallinity measured as the area under the DSC peak; as the crystalline fraction decreases (i.e., as the amorphous fraction increases), the Tₘ decreases.

A separate moisture absorption experiment was performed to confirm the crystallinity trend. The OLG1, OLG2 and OLG3 samples were placed within a closed chamber at 85% relative humidity (controlled by saturated salt solutions) at ambient temperature for 2 days. Afterwards, the amounts of moisture absorbed by the polymers were measured by thermogravimetric analysis (TGA). As shown in FIG. 2b (weight losses due to evaporation of water between about 60 and 100° C.), OLG1 had the greatest moisture content followed by OLG2, and OLG3 had the lowest moisture content; the moisture contents were almost comparable between OLG2 and OLG3. These results exactly agree with the DSC results (OLG1>OLG2≥OLG3 in amorphous fraction) (FIG. 2a).

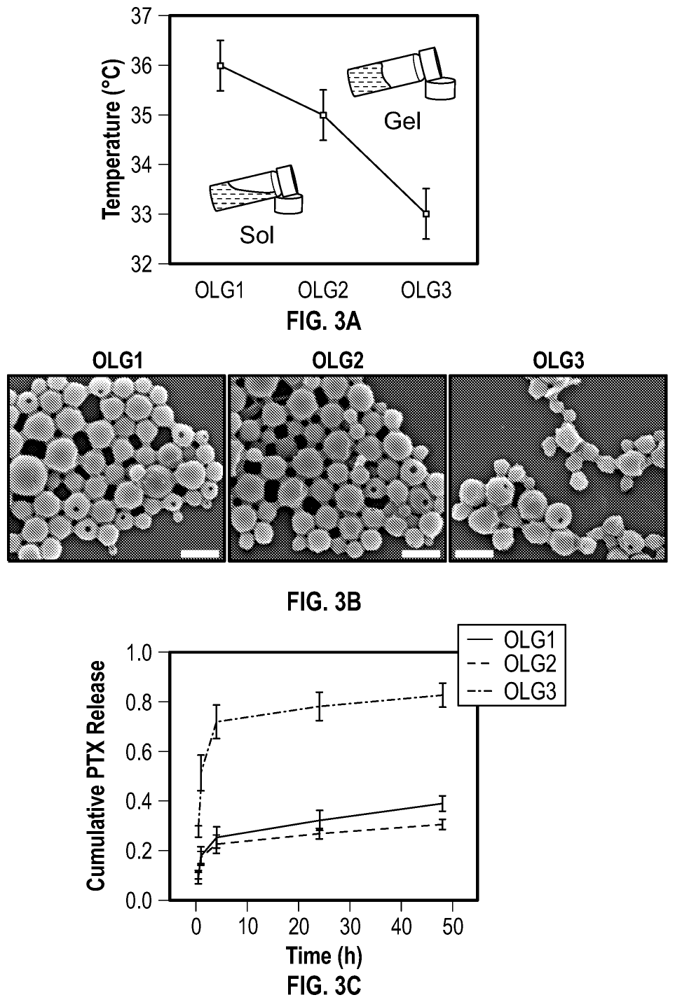

The effects of PLGA monomer sequence on PEG structures were visible even in aqueous self-assembly situations (i.e., in micelle solutions). Micelle solutions of OLG1, OLG2 and OLG3 were prepared by direct dissolution of the polymers (0.5% by weight) in Milli-Q water. Differences in the PEG corona properties of these micelles were examined by investigation of the interactions of these micelles with laponite (clay) nanoparticles in water. Gelation can also be induced in aqueous PEG-PLGA diblock solutions in the presence of added laponite nanoparticles, because PEG has strong affinity to laponite. Low molecular weight PEG homopolymers are effective at stabilizing laponite against gelation via adsorption and formation of a protective monolayer on the laponite surface, whereas higher molecular weight PEG chains (>˜1 kDa) typically form bridges between laponite particles, causing aggregation of the particles. The temperature-induced sol-gel transition behavior of aqueous mixtures containing 0.5% (by weight) OLG1, OLG2 or OLG3 micelles and 1.5% (by weight) laponite was examined by vial inversion. The results are presented in FIG. 3a. As shown in FIG. 3a, all OLG1/laponite, OLG2/laponite and OLG3/laponite suspensions underwent irreversible gelation at elevated temperatures. The sol-gel transition temperature was the highest for the OLG1/laponite system, second highest for the OLG2/laponite system, and lowest for the OLG3/laponite system, which appears to be consistent with a previous report that PLGA-PEG-PLGA triblock copolymers with longer LA/GL sequences exhibit lower gelation temperatures. The highest gelation temperature observed with the OLG1/laponite system is attributed to the lowest GL content near the block junction and thus the strongest interaction between the PEG corona chains and the PLGA core domain of the OLG1 micelles (among the three PEG-PLGA micelle/laponite systems analyzed); therefore, in this case, the interaction between the PEG and the laponite is the weakest, and the highest temperature is required to induced gelation, again clearly demonstrating the effect of PLGA block sequence uniformity on the self-assembly behavior of PEG-PLGA in water.

Lastly, the example explores how the monomer sequence distribution of the PLGA block affects the drug release properties of PEG-PLGA nanoparticles (NPs). Paclitaxel (PTX) was used as the drug of choice. Stable PTX-loaded PEG-PLGA nanoparticles (“PEG-PLGA/PTX NPs”) (having a hydrodynamic diameter of ˜700 nm) were prepared from the three different deblock polymers (OLG1, OLG2, and OLG3) by using the emulsion-evaporation method. The size (hydrodynamic diameter/polydispersity index) and composition (drug loading content/encapsulation efficiency) characteristics of PEG-PLGA/PTX NPs are summarized in Table 3 below. Scanning electron microscopy (SEM) images (FIG. 3b) show that all PEG-PLGA/PTX NPs generally have a spherical shape and smooth non-porous surfaces, although OLG3/PTX NPs appear to have a slightly more deformed structure with somewhat uneven surfaces. In all three formulations, single holes of ˜150 nm diameter were observed in about 10% of the particles, which have likely been created due to the evaporation flux of the organic solvent (DCM). Transmission electron microscopy (TEM) images indeed confirmed that these PEG-PLGA/PTX NPs are open hollow spheres.

TABLE 3
Size (hydrodynamic diameter/polydispersity index) and composition
(PTX encapsulation efficiency/loading content) characteristics
of OLG1/PTX, OLG2/PTX and OLG3/PTX NPs (prepared as described
in the Experimental Procedures section).

| | PEG-PLGA/PTX | PEG- | | |
|---|---|---|---|---|
| | NP Diameter | PLGA/PTX NP | | |
| Polymer | (nm) | PDI | PTX EE (%) | PTX LC (%) |
| OLG1 | 656.7 ± 8.8 | 0.130 ± 0.032 | 39.5 ± 0.9 | 3.9 ± 0.1 |
| OLG2 | 633.4 ± 8.3 | 0.147 ± 0.029 | 38.6 ± 3.8 | 3.9 ± 0.4 |
| OLG3 | 730.6 ± 11.1 | 0.159 ± 0.041 | 28.3 ± 0.6 | 2.8 ± 0.1 |

The PTX release kinetics of the three NP formulations were characterized at 37° C. (FIG. 3c). At regular intervals, 80% of the release medium (0.8 mL) was sampled after centrifugation and replaced with fresh PBS buffer containing 0.1% by volume of Tween 80; Tween 80 was added to accelerate the PTX release process. These time samples were analyzed for PTX concentration by HPLC. As shown in FIG. 3c, the PTX release was the fastest with OLG3(˜50% released immediately and ˜80% within 48 h). The release kinetics were comparable between OLG1 and OLG2, and they were much slower than that of OLG3 (only ˜30% released during the first 48 h in both the OLG1 and OLG2 cases) (FIG. 3a).

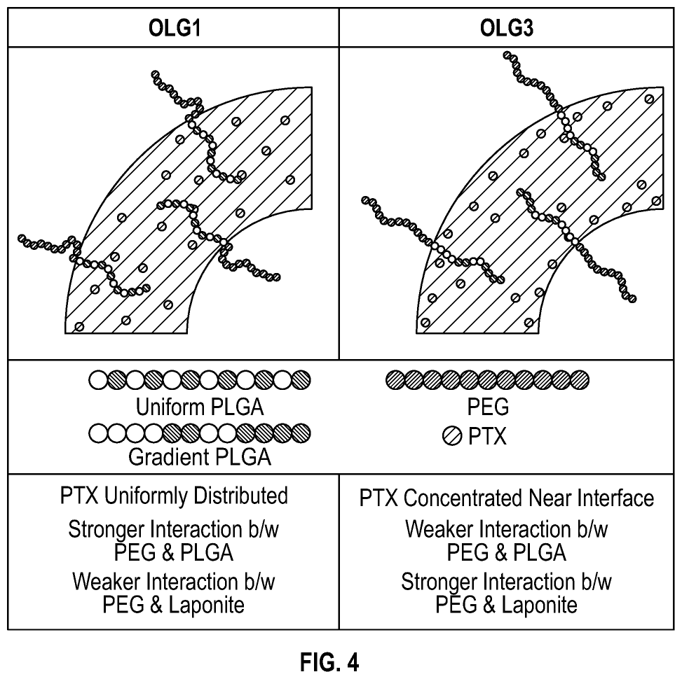

It is suspected that this dramatic difference in drug release kinetics was also caused by the difference in PLGA sequence characteristics. Based on the solubility parameter values of PLA and PGA (δ_{PLA}≅21.4 (J/cc)^{1/2}, and δ_{PGA}≅23.8 (J/cc)^{1/2}) and that of PTX (δ_{PTX}≅25.2 (J/cc)^{1/2}), the Flory-Huggins interaction parameters are estimated to be χ_{PLA/PTX}≅2.23 for PLA/PTX mixtures and χ_{PGA/PTX}≅0.24 for PGA/PTX mixtures at 25° C.; PTX is far more miscible with PGA than with PLA. Therefore, in PEG-PLGA NPs, PTX will partition more in the GL-rich region than in the LA-rich region. OLG3 contains longer GA sequences, particularly near the junction between the PEG and PLGA blocks. Therefore, the hydrophobic core domain of an OLG3/PTX NP must have a gradient in chemical composition; the region near the aqueous-core interface is rich with GL units, whereas the deeper side of the core domain is primarily composed of LA units. In the OLG3 case, because of its affinity to GL, PTX is also more concentrated in the peripheral region of the core domain, and as a result, PTX is released faster from the OLG3/PTX NPs than the other two systems (OLG1/PTX and OLG2/PTX NPs); in the latter cases, more uniform monomer sequence distribution in the PLGA block causes PTX to be also more homogeneously distributed within the core domain and thus to be released from it slower. These explanations are schematically summarized in FIG. 4.

In summary, the example shows development of a new, facile and scalable method (named the feed rate-controlled polymerization (FRCP) method), in which the LA+GL comonomer mixture is continuously fed into the DBU-catalyzed ROP at a sufficiently slow rate so that the large disparity of the reactivities of LA and GL does not bias the monomer sequence distribution of the copolymer product, and as a result, monodisperse “uniform PLGA” polymers with non-gradient sequence characteristics can easily be produced. Using this FRCP method, PEG-PLGA block copolymers (OLG1, OLG2, and OLG3) with varying degrees of PLGA sequence uniformity (ranging from more uniform PLGA (OLG1) to more gradient PLGA (OLG2 and OLG3)) have been prepared and used to demonstrate the effects of LA/GL sequence distribution on the interaction and drug release properties of the PEG-PLGA copolymers. As summarized in FIG. 4, the uniform LA/GL sequence distribution in the PEG-PLGA molecules renders the PEG chains less crystallizable in the neat state and less interactive with water and mineral surfaces in the aqueous self-assembled state. When drug molecules are loaded into a domain formed by uniform PLGA chains, the sequence uniformity forces the drug molecules to be more homogeneously distributed within the PLGA domain, which significantly suppresses the burst release of the drug and overall causes the release process to be slower and more sustained. The FRCP method offers a facile and scalable route for the production of non-gradient (precisely but) statistically monomer sequence-controlled substantially “uniform PLGA” materials for controlled release applications.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

### Exemplary Embodiments

The following exemplary embodiments are provided, the numbering of which is not to be construed as designating levels of importance:

Aspect 1 provides a method of preparing a substantially sequence-uniform aliphatic copolyester, comprising:
continuously contacting at least, a first monomer and a second monomer with an initiator and a catalyst to initiate ring-opening copolymerization of the first monomer and the second monomer, wherein
the first monomer and the second monomer are contacted with the initiator and catalyst at a feed rate that is slower than a polymerization rate of the first monomer and the second monomer.

Aspect 2 provides the method of Aspect 1, wherein the initiator and a catalyst are dispersed in a solvent or are free of a solvent.

Aspect 3 provides the method of any one of Aspects 1 or 2, wherein the first monomer and second monomer are present as a mixture prior to contact with the initiator and catalyst and dispersed in a solvent or the mixture is free of a solvent.

Aspect 4 provides the method of any one of Aspects 1 or 2, wherein the first monomer and the second monomer are located in separate containers prior to contact with the initiator and catalyst and are independently dispersed in a solvent or are free of a solvent.

Aspect 5 provides the method of any one of Aspects 1-4, wherein the first monomer is lactide (LA) and the second monomer is glycolide (GL).

Aspect 6 provides the method of any one of Aspects 1-5, wherein the first monomer is lactide (LA) and the second monomer is caprolactone (CL).

Aspect 7 provides the method of any one of Aspects 1-6, wherein the first monomer is glycolide (GL) and the second monomer is caprolactone (CL).

Aspect 8 provides the method of any one of Aspects 1-7, wherein the initiator is an alcohol.

Aspect 9 provides the method of Aspect 8, wherein the alcohol comprises one or more hydroxyl functional groups bonded to a carbon atom(s).

Aspect 10 provides the method of any one of Aspects 1-9, wherein the catalyst comprises an organic amidine compound, an organic guanidine compound, an aminopyridine compound, a thiourea compound, a heterocyclic carbene compound, a tin-containing compound, or a mixture thereof.

Aspect 11 provides the method of Aspect 10, wherein the organic amidine compound comprises 1,8-diazabicyclo[5.4.0]undec-7-ene (DBU).

Aspect 12 provides the method of any one of Aspects 10 or 11, wherein the organic guanidine compound comprises 1,5,7-triazabicyclo[4.4.0]dec-5-ene (TBD).

Aspect 13 provides the method of any one of Aspects 10-12, wherein the tin-containing compound comprises stannous octoate.

Aspect 14 provides the method of any one of Aspects 1-13, further comprising continuously contacting a third monomer with the initiator and the catalyst, wherein
the third monomer is contacted with the initiator and the catalyst, along with the first monomer and the second monomer at a feed rate that is slower than the polymerization rate of the first monomer, second monomer, and third monomer.

Aspect 15 provides the method of Aspect 14, wherein the first monomer, the second monomer, and the third monomer are present as a mixture of at least two of the first monomer, the second monomer, and the third monomer, prior to contact with the initiator and catalyst and dispersed in a solvent or the mixture is free of a solvent.

Aspect 16 provides the method of Aspects 15, wherein the first solvent, the second solvent, and the third solvent are located in separate containers prior to contact with the initiator and catalyst and are independently dispersed in a solvent or are free of a solvent.

Aspect 17 provides the method of any one of Aspects 1-16, wherein a molecular weight distribution polydispersity index of the aliphatic copolyester produced is in the range between 1.0 and 3.0.

Aspect 18 provides the method of any one of Aspects 1-17, wherein the aliphatic copolyester is poly(lactic-co-glycolic acid).

Aspect 19 provides the method of any one of Aspects 1-18, further comprising subjecting the produced aliphatic polyester to a pre- or post-polymerization PEGylation process.

Aspect 20 provides the method of any one of Aspects 2-19, wherein the solvent comprises dichloromethane.

Aspect 21 provides the method of any one of Aspects 1-20, wherein the comonomer feed rate is in a range of from about 5.0×10⁻⁶ moles of comonomers/min per mole of catalyst to about 5.0×10¹ moles of comonomers/min per mole of catalyst.

Aspect 22 provides the method of any one of Aspects 1-21, wherein the comonomer feed rate is in a range of from about 5.0×10⁻⁵ moles of comonomers/min per mole of catalyst to about 5.0 moles of comonomers/min per mole of catalyst.

Aspect 23 provides the method of any one of Aspects 1-22, wherein the monomers of the sequence-uniform aliphatic copolyester are substantially uniformly distributed.

Aspect 24 provides a nanoparticle comprising the aliphatic polyester of any one of Aspects 1-23.

Aspect 25 provides the nanoparticle of Aspect 24, wherein the nanoparticle has a substantially spherical shape.

Aspect 26 provides the nanoparticle of any one of Aspects 24 or 25, further comprising a pharmaceutical component distributed about the nanoparticle.

Aspect 27 provides the nanoparticle of any one of Aspects 24-26, wherein a diameter of the nanoparticle is in a range of from about 20 nm to about 2000 nm.

Aspect 28 provides the nanoparticle of any one of Aspects 24-27, wherein a diameter of the nanoparticle is in a range of from about 200 nm to about 2×10⁵ nm.

Aspect 29 provides a method of preparing a substantially sequence-uniform aliphatic copolyester, comprising:
providing two monomers (monomer 1 and monomer 2), wherein monomer 1 and monomer 2 are either mixed as a comonomer either in a solvent or without a solvent, or each independently either in a solvent or without a solvent;
providing an initiator and a catalyst either in a solvent or without a solvent;
adding monomer 1 and monomer 2 continuously into the initiator and catalyst mixture to initiate ring-opening copolymerization of the monomers, wherein monomer 1 and monomer 2 are added each at a sufficiently slow rate so that a disparity of the reactivities of monomer 1 and monomer 2 does not bias the monomer sequence distribution of the copolymer product.

Aspect 30 provides the method of Aspect 29, wherein monomer 1 is lactide (LA), and monomer 2 is glycolide (GL).

Aspect 31 provides the method of Aspect 29, wherein monomer 1 is lactide (LA), and monomer 2 is caprolactone (CL).

Aspect 32 provides the method of Aspect 29, wherein monomer 1 is glycolide (GL), and monomer 2 is caprolactone (CL).

Aspect 33 provides a method of preparing a substantially sequence-uniform aliphatic copolyester, comprising:
providing three monomers, lactide (LA), glycolide (GL), and caprolactone (CL), wherein LA, GL and CL are either mixed all together or in pairs as a comonomer either in a solvent or without a solvent, or each independently either in a solvent or without a solvent;
providing an initiator and a catalyst either in a solvent or without a solvent;
adding LA, GL and CL continuously into the initiator and catalyst mixture to initiate ring-opening copolymerization of the monomers, wherein LA, GL and CL are added each at a sufficiently slow rate so that a disparity of the reactivities of LA, GL and CL does not bias the monomer sequence distribution of the copolymer product.

Aspect 34 provides the method of Aspect 29 or 33, wherein the initiator is an alcohol, that is, a compound that carries at least one hydroxyl functional group (—OH) bound to a saturated carbon atom.

Aspect 35 provides the method of Aspect 29 or 33, wherein the catalyst is an organic amidine compound, such as 1,8-diazabicyclo[5.4.0]undec-7-ene (DBU).

Aspect 36 provides the method of Aspect 29 or 33, wherein the catalyst is an organic guanidine compound, such as 1,5,7-triazabicyclo[4.4.0]dec-5-ene (TBD).

Aspect 37 provides the method of Aspect 29 or 33, wherein the catalyst is a tin-containing compound, such as stannous octoate.

1. A method of preparing a substantially sequence-uniform aliphatic copolyester, comprising:
continuously contacting at least, a first monomer and a second monomer with an initiator and a catalyst to initiate ring-opening copolymerization of the first monomer and the second monomer, wherein
the first monomer and the second monomer are contacted with the initiator and the catalyst at a feed rate that is slower than a polymerization rate of the first monomer and the second monomer.

2. The method of claim 1, wherein the initiator and the catalyst are either dispersed in a solvent or free of a solvent.

3. The method of claim 1, wherein the first monomer and the second monomer are present as a mixture prior to contact with the initiator and the catalyst, and the mixture is dispersed in a solvent or the mixture is free of a solvent.

4. The method of claim 1, wherein the first monomer and the second monomer are located in separate containers prior to contact with the initiator and the catalyst, and are either independently dispersed in a solvent or free of a solvent.

5. The method of claim 1, wherein the first monomer is lactide (LA) and the second monomer is glycolide (GL).

6. The method of claim 1, wherein the first monomer is lactide (LA) and the second monomer is caprolactone (CL).

7. The method of claim 1, wherein the first monomer is glycolide (GL) and the second monomer is caprolactone (CL).

8. The method of claim 1, wherein the initiator is an alcohol.

9. (canceled)

10. The method of claim 1, wherein the catalyst comprises an organic amidine compound, an organic guanidine compound, an aminopyridine compound, a thiourea compound, a heterocyclic carbene compound, a tin-containing compound, or a mixture thereof.

11. The method of claim 10, wherein the organic amidine compound comprises 1,8-diazabicyclo[5.4.0]undec-7-ene (DBU).

12. The method of claim 10, wherein the organic guanidine compound comprises 1,5,7-triazabicyclo[4.4.0]dec-5-ene (TBD).

13. The method of claim 9, wherein the tin-containing compound comprises stannous octoate.

14. The method of claim 1, further comprising continuously contacting a third monomer with the initiator and the catalyst, wherein
the third monomer is contacted with the initiator and the catalyst, along with the first monomer and the second monomer at a feed rate that is slower than the polymerization rate of the first monomer, the second monomer, and the third monomer.

15. The method of claim 14, wherein the first monomer, the second monomer, and the third monomer are present as a mixture of at least two of the first monomer, the second monomer, and the third monomer, prior to contact with the initiator and the catalyst, and the mixture is dispersed in a solvent or the mixture is free of a solvent.

16. (canceled)

17. The method of claim 1, wherein the molecular weight distribution polydispersity index of the aliphatic copolyester produced is in the range between 1.0 and 3.0.

18. The method of claim 1, wherein the aliphatic copolyester is poly(lactic-co-glycolic acid).

19.-20. (canceled)

21. The method of claim 1, wherein the comonomer feed rate is in the range of approximately 5.0×10⁻⁶ moles of comonomers per minute per mole of catalyst to about 5.0×10¹ moles of comonomers per minute per mole of catalyst.

22.-23. (canceled)

24. A nano- or microparticle comprising the aliphatic polyester of claim 1.

25. The nano- or microparticle of claim 24, wherein the nano- or microparticle has a substantially spherical shape.

26. The nano- or microparticle of claim 24, further comprising a pharmaceutical component distributed about the nano- or microparticle.

27.-28. (canceled)
